(19) United States
(12) Patent Application Publication
(10) Pub. No.: US 2005/0196355 A1
(43) Pub. Date: Sep. 8, 2005

(54) Film products having controlled disintegration properties

(76) Inventors: Constantine Georgiades, East Brunswick, NJ (US); Seema Mody, Montville, NJ (US); Andre Soshinsky, Randolph, NJ (US); Zhen Zhang, Basking Ridge, NJ (US)

Correspondence Address: Warner-Lambert Company LLC, 201 Tabor Road, Morris Plains, NJ, 07950, US

(21) Appl. No.: 10/792,362

(22) Filed: Mar. 3, 2004

(51) Int. Cl. A61K 7/20; A61K 9/70

(52) U.S. Cl. 424/53; 424/443

(57) ABSTRACT

This invention relates generally to films having barrier as well as controlled disintegration properties and, more particularly, to controlled water disintegratable films.

### FIELD OF THE INVENTION

This invention relates generally to films having barrier as well as controlled disintegration properties and, more particularly, to controlled water disintegratable films.

### BACKGROUND OF THE INVENTION

A variety of topically-applied products, including strips, films, patches and the like, are known in the art. Such products are particularly useful where a protectant film are recommended or where drug or medication retention is desirable.

Film protectants are particularly desirable in situations where wounds or surface openings are present and must be protected. Alternatively, where a drug or medication is easily removed by rinsing or wiping the application area (e.g., transdermal applications), mechanical retention of the drug or medication becomes particularly desirable.

Most recently, strip or film type products have enjoyed renewed popularity in the oral care field. Particular interest has been paid to the areas of teeth whitening and oral transdermal delivery of drugs and medications.

Although a variety of strip or film type products have been disclosed, there still remains a need for improved film or film-like compositions which are easier to use and reduce the inconvenience or discomfort typically associated with the attachment of such foreign objects to sensitive parts of the body.

One disadvantage observed regarding the aforementioned film or strip products relates to the need of having to eventually peel off or in some other way remove and discard the film or strip product after delivery of the topical or systemic active.

In addressing this disadvantage, the inventors of the present invention have discovered that film compositions comprising select water insoluble polymers and a disintegration facilitator selected from the group consisting of a plasticizer, a water insoluble particulate or mixtures thereof provide film compositions having good protective properties as well as improved disintegration properties.

Accordingly an aspect of the present invention is to provide improved film products having protective properties such that the film prevents foreign substances, chemicals or actives from crossing from one side the film to the other.

Another aspect of the present invention is to provide film products having controlled (or an extended type or prolonged) disintegration or dissolution properties in aqueous environments.

Still one other aspect of the present invention is to provide film products for delivering topical or systemic actives.

Still yet one other aspect of the present invention is to provide film products for delivering topical or systemic actives wherein the film disintegrates within 60 minutes, optionally within 45 minutes, optionally, within 30 minutes or, optionally, within 15 minutes in an aqueous environment.

### SUMMARY OF THE INVENTION

The present invention relates to film compositions comprising at least one water insoluble polymer, a disintegration facilitator selected from the group consisting of a plasticizer, a water insoluble particulate or mixtures thereof and, optionally, at least one topical or systemic active wherein the film is partially, substantially or completely disintegratable in an aqueous environment. The film composition of the present invention can be used as a single layer film or in conjunction with one or more additional film layers to form a bi- or multi-layered film product.

In one embodiment, the film of the present invention can be in the form of a single layer film comprising an adhesive composition wherein the adhesive composition comprises an adhesive substance and a topical or systemic active. The film is then applied to the teeth, mucosa or other affected area of the skin or mouth and allowed to disintegrate over time in the presence of oral fluids or other aqueous media.

In another embodiment, the film of the present invention forms the first or backing layer of a bi-layer film where the second layer is a water soluble polymer film layer such as that described in U.S. Pat. No. 6,596,298 to Leung et al. and U.S. Pat. No. 6,419,903 to Xu et al., both of which are herein incorporated by reference in their entirety. The bilayer film is then applied to the teeth, oral mucosa or other affected area of the skin or mouth and allowed to disintegrate over time in the presence of oral fluids or other aqueous media.

Similarly, the film of the present invention may be incorporated in multi-layer films and used as above to achieve the benefits of the present invention.

Methods of using the above film compositions are also disclosed.

### DETAILED DESCRIPTION OF THE PRESENT INVENTION

The film compositions of the present invention can comprise, consist of, or consist essentially of the essential elements and limitations of the invention described herein, as well any of the additional or optional ingredients, components, or limitations described herein.

All percentages, parts and ratios are based upon the total weight of the wet film composition of the present invention, unless otherwise specified. All such weights as they pertain to the listed ingredients are based on the active level and, therefore, do not include carriers or by-products that may be included in commercially available materials, unless otherwise specified.

The term “safe and effective amount” as used herein means an amount of a compound or composition such as a topical or system active sufficient to significantly induce a positive benefit, for example, a teeth whitening, antimicrobial and/or analgesic benefit, including independently the benefits disclosed herein, but low enough to avoid serious side effects, i.e., to provide a reasonable benefit to risk ratio, within the scope of sound judgment of the skilled artisan.

The term “adhesive” as used herein, means any material or composition that is capable of sticking to the site of topical application or administration and includes, but is no limited to, mucoadhesives, pressure-sensitive adhesive (adheres upon application of pressure), moistenable adhesives (adheres in the presence of water) and tacky or sticky type adhesives (adheres upon immediate contact with a surface).

The term “foreign substances” as used herein means dirt, infectious microorganisms and the like.

Optionally, the film compositions of the present invention are clear. The term “clear” as defined herein ranges from transparent to translucent as observed with the naked eye.

The film compositions of the present invention, including the essential and optional components thereof, are described in detail hereinafter.

The Water Insoluble Polymer

The film compositions of the present invention comprise water insoluble polymers. Suitable water insoluble polymers include, but are not limited to, hydrogenated vegetable oils; natural rosins such as wood rosins and gum rosins; vegetable proteins such as corn protein, pea protein or soy protein; hydrogenated caster oil; polyvinyl chloride; shellac; polyurethane; cellulose derivatives such as cellulose or ethylcellulose; waxes; polymers such as those sold under the Trade Mark Eudragit RS or a mixture thereof.

Hydrogenated vegetable oils suitable for use herein include, but are not limited to, hydrogenated forms of safflower oil, caster oil, coconut oil, cottonseed oil, canola oil, menhaden oil, palm kernel oil, palm oil, peanut oil, soybean oil, rapeseed oil, linseed oil, rice bran oil, pine oil, sesame oil, sunflower seed oil, hydrogenated safflower oil and mixtures thereof.

Examples of the waxes suitable for use herein include, but are not limited to, paraffin, carnauba wax, candelilla wax, sugarcane wax, beeswax, cetyl esters wax, montan wax, glycowax, castor wax, spermaceti wax, shellac wax, microcrystalline wax, vaseline and mixtures thereof.

Eudragit polymers are polymeric lacquer substances based on acrylate and methacrylate. Polymers sold under the Trademark Eudragit RL and RS are resins comprising copolymers of acrylic and methacrylic acid esters with a low content of quaternary ammonium groups and are described in the “Eudragit” brochure of Rohm Pharma GmbH (1982) wherein detailed physical-chemical data of these products is given. The ammonium groups are present as salts and give rise to permeability of the lacquer films. Eudragit RL and RS are freely permeable (RL) or slightly permeable (RS), respectively independent of pH. Additional water insoluble polymers are detailed in U.S. Pat. Nos. 6,183,777; 4,721,619; and 6,251,427, each of which are herein incorporated by reference in their entirety.

Mixtures of any of the above ingredients can also be used.

In certain embodiment, the water insoluble polymer can includes shellac sold under the name Pharmaceutical Glaze and supplied by Mantrose Haeser Co., Attleboro, Ma.

When incorporated in the film compositions of the present invention, the water insoluble polymer is present at a concentration of from about 10% to about 80%, optionally, from about 15% to about 40%, and, optionally, from about 20% to about 35%, by weight, of the wet film composition.

The Disintegration Facilitator

Plasticizers or Plasticizing Agents

The film compositions of the present invention also comprise at least one disintegration facilitator selected from the group consisting of plasticizers or plasticizing agents, water insoluble particles or mixtures thereof.

Examples of suitable plasticizers include, but are not limited to, citric acid alkyl esters, glycerol esters such as glycerol monooleate and glycerol monostearate, phthalic acid alkyl esters, sebacic acid alkyl esters, sucrose esters, sorbitan esters, acetylated monoglycerides, glycerols, fatty acid esters, glycols, propylene glycol, and polyethylene glycols 200 to 12,000 and mixtures thereof. Specific plasticizers include, but are not limited to, lauric acid, sucrose, sorbitol, triethyl citrate, acetyl triethyl citrate, triacetin (glyceryl triacetate), poloxamers, alkyl aryl phosphates, diethyl phthalate, tributyl citrate, dibutyl phthalate, dibutyl sebacate, polysorbate, Carbwax® series of polyethylene glycols (Union Carbide Corporation) and mixtures thereof.

In certain embodiments, the plasticizers can include mono- and di-glycerides of edible fats or oils supplied by Lonza Inc., Fair Lawn, N.J. or Eastman Triacetin (food grade) supplied by Eastman Chemical Company, Kingsport, Tenn.

When incorporated in the film compositions of the present invention, the plasticizer is present at a concentration of from about 0.1% to about 10%, preferably from about 0.1% to about 5%, and most preferably from about 0.5% to about 1.5% by weight of the wet film composition.

Water Insoluble Particles

The disintegration facilitator can also be a water insoluble particle. Various kinds of organic powders and inorganic powders can be used as the water-insoluble particles.

The inorganic powders which are useful herein include, but are not limited to, microfine particles or granules of alumina, talc, magnesium stearate, titanium dioxide, barium titanate, magnesium titanate, calcium titanate, strontium titanate, zinc oxide, silica sand, clay, mica, tabular spar, diatomaceous earth, various inorganic oxide pigments, chromium oxide, cerium oxide, iron red, antimony trioxide, magnesium oxide, zirconium oxide, barium sulfate, barium carbonate, calcium carbonate, silica (colloidal or fumed), silicon carbide, silicon nitride, boron carbide, tungsten carbide, titanium carbide, carbon black and mixtures thereof.

The organic powders which are useful herein include cross-linked and non-cross-linked polymer powders, organic pigments, charge controlling agents, and waxes, for example. The cross-linked and non-cross-linked resin powders include, but are not limited to, resin powders of the styrene type, acrylic type, methacrylic type, polyethylene type, polypropylene type, silicone type, polyester type, polyurethane type, polyamide type, epoxy type, polyvinyl butyral type, rosin type, terpene type, phenol type, melamine type, and guanamine type, for example. Mixtures of any of the above organic or inorganic powders can also be used. Additional particles useful in the present invention can be found in U.S. Pat. No. 6,475,500; U.S. Pat. No. 5,611,885; and U.S. Pat. No. 4,847,199 each of which are herein incorporated by reference in its entirety.

The water insoluble particles of the present invention generally have a particle size of less than 10 microns, optionally, from about 0.01 microns to about 5 microns, optionally, from about 0.1 microns to about 1 micron, and, optionally, from about 0.1 to about 0.5 microns.

In certain embodiments, the insoluble particles can include Cabosil M-5 (fumed untreated silica) supplied by Cabot, Tuscola, Ill.

When incorporated in the film compositions of the present invention, the water insoluble particle is present at a concentration of from about 0.1% to about 20%, optionally, from about 0.5% to about 15%, and, optionally, from about 1% to about 10% by weight of the wet film composition.

Optional Ingredients

Various topical and systemic actives can also be incorporated into the films of the present invention. The term “topical or system active” as used herein includes curative, prophylactic and cosmetic active substances or compositions thereof. Examples of the conditions these substances may address include, but are not limited to one or more of, appearance and structural changes to teeth, whitening, stain bleaching, stain removal, plaque removal, tartar removal, cavity prevention and treatment, inflamed and/or bleeding gums, mucosal wounds, lesions, ulcers, aphthous ulcers, cold sores, tooth abscesses, tooth and/or gum pain, tooth sensitivity (e.g. to temperature changes), and the elimination of mouth malodour resulting from the conditions above and other causes such as microbial proliferation. Additionally, the films of the present invention are useful for treating and/or preventing wounds, lesions, ulcers, cold sores and the like of the lips and skin generally.

Suitable topical actives for use in and around the oral cavity include any substance that is generally considered as safe for use in the oral cavity and that provides a change to the overall health of the oral cavity. The level of topical oral care active in the present invention may generally be from about 0.01% to about 40% or, optionally, from about 0.1% to 20% by weight of the wet film.

The topical oral care actives of the present invention may include many of the actives previously disclosed in the art. The following is a non all-inclusive list of oral care actives that may be used in the present invention.

Essential oils may be included in or associated with the films the present invention. Essential oils suitable for use herein are described in detail in U.S. Pat. No. 6,596,298 to Leung et al., previously incorporated by reference in its entirety.

Teeth whitening actives may be included in the films of the present invention. The actives suitable for whitening are selected from the group consisting of oxalates, peroxides, metal chlorites, perforates, percarbonates, peroxyacids, and mixtures thereof. Suitable peroxide compounds include: hydrogen peroxide, calcium peroxide, sodium peroxide, carbamide peroxide, urea peroxide, sodium percarbonate and mixtures thereof. Optionally, the peroxide is hydrogen peroxide. Suitable metal chlorites include calcium chlorite, barium chlorite, magnesium chlorite, lithium chlorite, sodium chlorite and potassium chlorite. Additional whitening actives may be hypochlorite and chlorine dioxide. A preferred chlorite is sodium chlorite. The effectiveness of whitening actives can, optionally, be enhanced by means of a catalyst, i.e. a two-component peroxide-catalyst; system. Useful whitening agent catalysts or catalytic agents can be found in U.S. Pat. No. 6,440,396 to McLaughlin, Gerald, herein incorporated by reference in its entirety.

When incorporating peroxide actives, the film compositions of the present invention can, optionally, contain peroxide active stabilizers. Peroxide active stabilizers suitable for use herein include, but are not limited to polyethylene glycols such as PEG 40 or PEG 600; zinc salts such as zinc citrate; polyoxyalkylene block-polymers (e.g., Pluronics); aminocarboxylic acids or salts thereof; glycerols; dyes such as Blue #1 or Green #3; phosphates such as phosphoric acid, sodium phosphate or sodium acid pyrophosphate; stannous salts such as stannous chloride; sodium stannate; citric acid; etidronic acid; carbomers or carboxypolymethylenes such as those of the Carbopol® seriers, butylated hydroxytoluene (BHT), ethylenediaminetetraacetic acid (EDTA) and mixtures thereof.

Anti-tartar agents useful herein include: phosphates. Phosphates include pyrophosphates, polyphosphates, polyphosphonates and mixtures thereof. Pyrophosphates are among the best known for use in dental care products. Pyrophosphate ions delivered to the teeth derive from pyrophosphate salts. The pyrophosphate salts useful in the present compositions include the dialkali metal pyrophosphate salts, tetra-alkali metal pyrophosphate salts, and mixtures thereof. Disodium dihydrogen pyrophosphate (Na₂H₂P₂O₇), tetrasodium pyrophosphate (Na₄P₂O₇), and tetrapotassium pyrophosphate (K₄P₂O₇) in their unhydrated as well as hydrated forms are preferred. Anticalculus phosphates include potassium and sodium pyrophosphates; sodium tripolyphosphate; diphosphonates, such as ethane-1-hydroxy-1,1-diphosphonate; 1-azacycloheptane-1,1-diphosphonate; and linear alkyl diphosphonates; linear carboxylic acids and sodium and zinc citrate.

Agents that may be used in place of or in combination with the pyrophosphate salt include materials such as synthetic anionic polymers including polyacrylates and copolymers of maleic anhydride or acid and methyl vinyl ether (e.g. Gantrez, as described, for example, in U.S. Pat. No. 4,627,977, to Gaffar et al. herein incorporated by reference in its entirety, as well as e.g. polyamino propane sulfonic acid (AMPS), zinc citrate trihydrate, polyphosphates (e.g. tripolyphosphate; hexametaphosphate), diphosphonates (e.g. EHDP, AMP), polypeptides (such as polyaspartic and polyglutamic acids), and mixtures thereof.

One of more fluoride ion sources incorporated into the film compositions as anticaries agents. Fluoride ions are included in many oral care compositions for this purpose, and similarly may be incorporated in the invention in the same way. Detailed examples of such fluoride ion sources can be found in U.S. Pat. No. 6,121,315 to Nair et al., herein incorporated by reference in its entirety.

Also useful herein are tooth desensitizing agents. Tooth desensitizing agents that may be used in the present invention include potassium nitrate, citric acid, citric acid salts, strontium chloride, and the like, as well as other desensitizing agents known in the art. The amount of desensitizing agent included within the dental whitening compositions of the present invention may vary according to the concentration of the potassium nitrates, the desired strength and intended treatment times. Accordingly, if included at all, the other desensitizing agents will preferably be included in an amount in a range from about 0.1% to about 10% by weight of the dental desensitizing composition, more preferably in a range from about 1 to about 7% by weight of the wet film composition.

Antimicrobial agents can also be present in the film compositions of the present invention as oral agents or topical skin and/or systemic actives. Such agents may include, but are not limited to, 5-chloro-2-(2,4-dichlorophenoxy)-phenol, commonly referred to as triclosan, chlorhexidine, alexidine, hexetidine, sanguinarine, benzaLkonium chloride, salicylamide, domiphen bromide, cetylpyridium chloride (CPC), tetradecyl pyridinium chloride (TPC); N-tetradecyl-4-ethyl pyridinium chloride (TDEPC); octenidine; delmopinol, octapinol, and other piperidino derivatives, niacin preparations; zinc/stannous ion agents; antibiotics such as AUGMENTIN, amoxyicillin, tetracycline, doxycyline, minocycline, and metronidazole; and analogs, derivatives and salts of the above antimicrobial agents and mixtures thereof.

Anti-inflammatory agents can also be present in the film compositions of the present invention as oral agents or topical skin and/or systemic actives. Such agents may include, but are not limited to, non-steroidal anti-inflammatory agents or NSAIDs, such as propionic acid derivatives; acetic acid derivatives; fenamic acid derivatives; biphenylcarboxylic acid derivatives; and oxicams. All of these NSAIDS are fully described in U.S. Pat. No. 4,985,459 to Sunshine et al., issued Jan. 15, 1991, incorporated by reference herein in its entirety. Examples of useful NSAIDS include acetyl salicylic acid, ibuprofen, naproxen, benoxaprofen, flurbiprofen, fenoprofen, fenbufen, ketoprofen, indoprofen, pirprofen, carprofen, oxaprozin, pranoprofen, microprofen, tioxaprofen, suprofen, alminoprofen, tiaprofenic acid, fluprofen, bucloxic acid and mixtures thereof. Also useful are the steroidal anti-inflammatory drugs such as hydrocortisone and the like, and COX-2 inhibitors such as such as meloxicam, celecoxib, rofecoxib, valdecoxib, etoricoxib or mixtures thereof. Mixtures of any of the above anti-inflammatories may be used.

Anesthetic agent may also be incorporated herein. Examples of suitable anesthetic agents include, but are not limited to, benzocaine, betoxycaine, biphenamine, bupivacaine, butacaine, dibucaine hydrochloride, dyclonine, lidocaine, mepivacaine, procaine, propanidid, propanocaine, proparacaine, propipocaine, propofol, propoxycaine hydrochloride, pseudococaine, tetracaine hydrochloride and mixtures thereof.

Upper respiratory actives can also be used herein. Examples of such actives are sympathomimetic agents administered systemically or topically for decongestant use, including propylhexedrine, phenylephrine, phenylpropanolamine, pseudoephedrine, naphazoline hydrochloride, oxymetazoline hydrochloride, tetrahydrozoline hydrochloride, xylometazoline hydrochloride, and ethylnorepinephrine hydrochloride; anti-histamines are chlorpheniramine, brompheniramine, clemastine, ketotifen, azatadine, loratadine, terfenadine, cetirizine, astemizole, tazifylline, levocabastine, diphenhydramine, temelastine, etolotifen, acrivastine, azelastine, ebastine, mequitazine, mizolastine, levocetirizine, mometasone furoate, carebastine, ramatroban, desloratadine, noberastine, selenotifen, alinastine, efletirizine, tritoqualine, norastemizole, tagorizine, epinastine, acrivastine and mixtures thereof; antitussives such as dextromethorphan, benzonatate, and guifenecin and mixtures thereof. Other useful upper respiratory actives and be found in U.S. Pat. No. 4,619,934, herein incorporated by reference in its entirety.

Gastro-intestinal actives can also be incorporated. Examples of suitable gastro-intestinal actives include anticholinergics, including: atropine, clidinium and dicyclomine; antacids, including aluminum hydroxide, basic bismuth salts such as bismuth subsalicylate, bismuth ranitidine citrate, bismuth subcitrate, bismuth subnitrate, aluminum or bismuth salts of polysulfated saccharides such as aluminum sucrose octasulfate or bismuth sucrose octasulfate, simethicone, calcium carbonate and magaldrate (other examples of antacids can be found in 21CFR 331.11 which is incorporated herein by reference); H (2)-receptor antagonists, including cimetidine, famotidine, nizatidine and ranitidine; laxatives, including: bisacodyl, picosulfate, and casanthrol (other examples of laxatives can be found in the Federal Registry, Vol. 50, No. 10, Jan. 15, 1985, pp. 2152-58, which is incorporated herein by reference); gastroprotectants, including sucralfate and sucralfate humid gel; gastrokinetic and prokinetic agents including cisapride, metoclopramide and eisaprode; proton pump inhibitors including omeprazole, lanzoprazole, and antidiarrheals including: diphenoxylate and loperamide; agents which are bacteriostatic or bactericidal to the ulcer-inducing organism Heliobacter pylori such as amoxicillin, metronidazole, erythromycin, or nitrofurantoin and others agents for treating H. pylori disclosed in U.S. Pat. No. 5,256,684, which is incorporated herein by reference in its entirety; polyanionic materials useful for the treatment of ulcers and other gastrointestinal disorders including amylopectin, carragemum, sulfated dextrins, inositol hexaphosphate, or other similar agents and mixtures thereof.

Nutrients may improve the condition of the oral cavity and can be included in the oral care substances or compositions of the present invention. Examples of nutrients include minerals, vitamins, oral nutritional supplements, enteral nutritional supplements, and mixtures thereof.

Smoking cessation agents such as nicotine may also be incorporated in the film compositions of the present invention.

An individual enzyme or combination of several compatible enzymes can also be included in the oral care substance or composition of the present invention.

Enzymes are biological catalysts of chemical reactions in living devices. Enzymes combine with the substrates on which they act forming an intermediate enzyme substrate complex. This complex is then converted to a reaction product and a liberated enzyme which continues its specific enzymatic function.

Enzymes provide several benefits when used for cleansing of the oral cavity. Proteases break down salivary proteins which are absorbed onto the tooth surface and form the pellicle; the first layer of resulting plaque. Proteases along with lipases destroy bacteria by lysing proteins and lipids which form the structural component of bacterial cell walls and membranes. Dextranases break down the organic skeletal structure produced by bacteria that forms a matrix for bacterial adhesion. Proteases and amylases, not only present plaque formation, but also prevent the development of calculus by breaking-up the carbohydrate protein complex that binds calcium, preventing mineralisation. Enzymes useful in the present invention include any of the commercially available proteases, glucanohydrolases, endoglycosidases, amylases, mutanases, lipases and mucinases or compatible mixtures thereof. Preferred are the proteases, dextranases, endoglycosidases and mutenases, most preferred being papain, endoglycidase, lysozyme or a mixture of dextranase and mutanase.

Other materials that can be used with the present invention include commonly known mouth and throat products. These products include, but are not limited to anti-fungal, antibiotic and analgesic agents. Antioxidants are generally recognized as useful in compositions such as those of the present invention. Antioxidants that may be included in the oral care composition or substance of the present invention include, but are not limited to Vitamin E, ascorbic acid, Uric acid, carotenoids, Vitamin A, flavonoids and polyphenols, herbal antioxidants, melatonin, aminoindoles, lipoic acids and mixtures thereof.

Histamine-(H-2) receptor antagonist compounds (H-2 antagonists) may be used in the oral care composition of the present invention. As used herein, selective H-2 antagonists are compounds that block H-2 receptors, but do not have meaningful activity in blocking histamine-(H-1) receptors.

Additional useful actives can be found in U.S. Pat. No. 6,638,528 herein incorporated by reference in its entirety.

An additional carrier material may also be added to the film composition of the present invention. These materials can be added as additional components for properties other than those previously mentioned and can include humectants and include glycerin, sorbitol, polyethylene glycol and the like. The film composition may comprise the substance itself, together with one or more substance enhancers, for example catalysts and/or potentiators to modify the release and/or activity of the substance.

The film compositions of the invention may additionally comprise additional substances such as flavours, colours, etc. which may for example be deposited onto the surface of the film or impregnated into the bulk of the film. The topical or system active is preferably teeth whitening substance. The teeth whitening substance can take the form of a peroxide-containing gel. Suitable gels may be based on glycerol containing a peroxide such as hydrogen peroxide or an organic peroxide. A suitable gel is that disclosed in U.S. Pat. No. 3,657,413, for example that sold under the trade mark PROXIGEL by The Block Drug Company (USA) (since acquired by GlaxoSmithKline plc). Other suitable peroxide-containing gels are for example disclosed in the art references cited above. The film may have the topical or system active deposited upon its surface.

A pH adjusting agent may also be added to optimise the storage stability of the gel and to make the substance safe for the oral tissues. These pH adjusting agents, or buffers, can be any material which is suitable to adjust the pH of the oral care substance. Suitable materials include sodium bicarbonate, sodium phosphate, sodium hydroxide, ammonium hydroxide, sodium stannate, triethanolamine, citric acid, hydrochloric acid, sodium citrate, and combinations thereof. The pH adjusting agents are added in sufficient amounts so as to adjust the pH of the substance or composition to a suitable value, e.g. about 4.5 to about 11, preferably from about 5.5 to about 8.5, and more preferably from about 6 to about 7. The pH adjusting agents are generally present in an amount of from about 0.01% to about 15% and preferably from about 0.05% to about 5%, by weight of the oral care substance.

For example a gel may be deposited directly as a layer on a surface of a film layer as described above. Alternatively a gel may be absorbed into the above-described film layer, or impregnated into the bulk of the film material, or deposited between layers of a multiple layered film.

Methods of depositing substances upon the surfaces of film materials as described above are known, for example printing, e.g. silo screen printing, passing between impregnated rollers, dosing, a pump and nozzle, spraying, dipping etc. Methods of impregnating substances into the bulk of film materials are also known, for example admixing the substance into the strip material and then forming the strip, or exposure of the strip to the substance under conditions which cause the substance to be impregnated into the strip. Alternatively, one example of the film material may be a foam material, particularly an open-cell foam material, and the substance may be impregnated into the strip material by introducing the substance into the cells of the foam.

In one another embodiment, the film of the present invention forms the first or backing layer of a bilayer where as the second layer is a water soluble polymer film layer such as that described in U.S. Pat. No. 6,596,298 to Leung et al. and U.S. Pat. No. 6,419,903 to Xu et al., both of which are herein incorporated by reference in their entirety. The bilayer film is then applied to the teeth, oral mucosa or other affected area of the skin or mouth and allowed to disintegrate over time in the presence of saliva or other aqueous media.

The device of the invention may be marked with one or more visible symbol, e.g. text matter, a trade mark, a company logo, an area of color, or an alignment feature such as a visible line or notch etc. to assist the user in applying the device to the teeth in a proper alignment. Such an alignment feature may for example comprise a symbol to show the user which way up the device should be whilst applying the device to the teeth, or which of a pair of the devices is intended for the upper teeth and which for the lower teeth. This way the device may be made more visually attractive and/or easier to use. Such symbol(s) may be applied by conventional printing or embossing processes, e.g. silk screen printing, inkjet printing etc. to the surface of the plastically deformable material opposite to the surface on which is attached the layer of an absorbent material.

If such a visible symbol is applied to this surface, a cover layer can, optionally, be applied over the symbol, for example to protect it. This cover layer may be transparent or translucent to allow visible symbols to be seen through this layer. Such a cover layer can, optionally, be applied to the film by pressing, e.g. rolling, the material of the cover layer in contact with the film.

Methods for Delivering Topical and Systemic Actives

The present invention can be used where retention of the topical or systemic active is required for topical activity or adequate systemic absorption. The film compositions of the present invention are particularly useful for whitening tooth surfaces. Generally, the delivery of the teeth whitening actives involves topically applying the inventive film containing a safe and containing effective amount of such actives to a tooth or teeth and gums in a manner described in U.S. Pat. Nos. 5,894,017; 5,891,453; 6,045,811; and 6,419,906, each of which is herein incorporated by reference in its entirety. The frequency of application and the period of use will vary widely depending upon the level of treatment required or desired, e.g., the degree of teeth whitening and/or degree of topical wound healing/disinfection desired.

When applied as a patch for the skin or mucosa, the films of the present invention can be useful for problem skin areas needing more intensive treatment or for the transderamal delivery of drugs. The patch can be occlusive, semi-occlusive or non-occlusive. The topical or systemic actives of the present invention can be contained within or coated on the surface of the film or be applied to the skin prior to application of the film. Additionally, the film can be applied wet to form a film when dried on the area of application. The film can also include actives such as chemical initiators for exothermic reactions such as those described in PCT application WO 9701313 to Burkett et al. Optionally, the film can be applied at night as a form of night therapy. Examples of useful transdermal systems are described in U.S. Pat. Nos. 3,598,122; 3,598,123; 3,731,683; 3,797,494; 4,286,592; 4,314,557; 4,379,454; 4,435,180; 4,559,222; 4,568,343; 4,573,999; 4,588,580; 4,645,502; 4,704,282; 4,816,258; 4,849,226; 4,908,027; 4,943,435; and 5,004,610, all of which are herein incorporated by reference in their entirety. Actives commonly associated with transdermal delivery are disclosed in U.S. Pat. Nos. 5,843,468 and 5,853,751, both of which are herein incorporated by reference in their entirety.

### EXAMPLES

The film compositions illustrated in following examples illustrate specific embodiments of the film compositions of the present invention, but are not intended to be limiting thereof. Other modifications can be undertaken by the skilled artisan without departing from the spirit and scope of this invention.

All exemplified film compositions can be prepared by conventional formulation and mixing techniques. Component amounts are listed as weight percents and exclude minor materials such as diluents, filler, and so forth. The listed formulations, therefore, comprise the listed components and any minor materials associated with such components.

### Example I

The following is an example of a stand alone film of the present invention

| | AMOUNT |
|---|---|
| INGREDIENT | (weight percent) |
| DISTILLED WATER | 10.00 |
| ISO-PROPYL ALCOHOL | 79.00 |
| SILICA (fumed untreated)¹ | 4.00 |
| GLYCERIN USP SPECIAL | 2.00 |
| ZEIN² | 5.00 |

¹Supplied under the tradename Cabosil ® by Cabot, Tuscola, Ill.
²Protein from Corn, (Supplied by Freeman Industries, Tuckahoe, NY).

In suitable beaker, zein, silica, alcohol, glycerin and water are mixed until uniform and homogenous.

The contents of the beaker is then cast at desired thickness on a non-stick surface or sheet at room temperature to form the inventive.

### Example II

The following is an example of a stand alone film of the present invention

| | AMOUNT |
|---|---|
| INGREDIENT | (weight percent) |
| ALCOHOL USP/EP | 38.00 |
| SILICA (fumed untreated)¹ | 2.00 |
| CAPOL 150² | 60.00 |

¹Supplied under the tradename Cabosil ® by Cabot, Tuscola, Ill.
²Contains Ethanol, Shellac, Hydrogenated Vegetable Oil (Coconut Origin) (Supplied by Centerchem, Inc., Norwalk, CT).

In suitable beaker, Capol 150, silica, and alcohol are mixed until uniform and homogenous.

The contents of the beaker is then cast at desired thickness on a non-stick surface or sheet at room temperature to form the inventive.

### Example III

The following is an example of a paint-on film forming composition of the present invention

| | AMOUNT |
|---|---|
| INGREDIENT | (weight percent) |
| PHARMACEUTICAL GLAZE¹ | 56.00 |
| MAGNESIUM STEARATE² | 3.00 |
| TRIACETIN³ | 1.00 |
| ALCOHOL USP/EP | 40.00 |

¹Shellac supplied by Mantrose Haeser Co., Attleboro, Ma.
²Magnesium stearate, Hyqual, vegetable source supplied by Mallinckrodt Chemicals, Phillipsburg, NJ.
³Eastman Triacetin (food grade) supplied by Eastman Chemical Company, Kingsport, TN.

In suitable beaker, Pharmaceutical Glaze, magnesium stearate, triacetin, and alcohol are mixed until uniform and homogenous.

The contents of the beaker is then placed in a suitable air-tight container for later application to the skin, teeth, or oral mucosa by the consumer as a paint-on film.

### Example IV

The following is an example of a bi-layer, teeth whitening film of the present invention.

| | |
|---|---|
| | AMOUNT |
| INGREDIENT | (weight percent) |

Adhesive Layer

| | | |
|---|---|---|
| XANTHAN GUM¹ | 0.0174% | w/w |
| LOCUST BEAN GUM, CLARIFIED² | 0.0348% | w/w |
| CARRAGEENAN³ | 0.1740% | w/w |
| PULLULAN⁴ | 4.1000% | w/w |
| POVIDONE, USP K-90⁵ | 12.4000% | w/w |
| SUCRALOSE⁶ | 0.7000% | w/w |
| POTASSIUM PHOSPHATE MONOBASIC NF | 0.0700% | w/w |
| PURIFIED WATER, USP/EP | 72.4948% | w/w |
| HYDROGEN PEROXIDE 35%⁷ | 5.7100% | w/w |
| FLAVOR | 2.5890% | w/w |
| POLYSORBATE 80 NF/EP⁸ | 0.3550% | w/w |
| EMULSIFIER⁹ | 0.3550% | w/w |
| GLYCERIN USP SPECIAL | 1.0000% | w/w |

Backing Layer

| | | |
|---|---|---|
| PHARMACEUTICAL GLAZE, 4-LB CUT NF¹⁰ | 55.0000% | w/w |
| SILICA¹¹ (fumed untreated) | 4.0000% | w/w |
| ALCOHOL USP/EP | 40.0000% | w/w |
| GLYCERYL STEARATE SE¹² | 1.0000% | w/w |

¹Supplied under the name Keltrol T by CP Kelco, Chicago, IL
²Sold under the name Viscogum BCR 20/80 by Degussa Texturant Systems, Atlanta, GA
³Supplied under the name Viscarin SD339 by FMC Biopolymer, Philadelphia, PA.
⁴PI-20 grade supplied by Hayashibara.
⁵Polyvinylpyrrolidone, USP K-90, International Specialties Products(ISP), Wayne, NJ.
⁶ALB CG 35% hydrogen peroxide solution, Atofina, Philadelphia, Pa.
⁷Supplied under the tradename Splenda ®, by McNeil Pharmaceuticals, NewBrunswick, NJ.
⁸Tween 80, supplied by Quest, Hoffmann Estates, Ill.
⁹Mixture of mono- and di-oleates supplied under name Atmos 300 by American Ingredients, Kansas City, Mo.
¹⁰Shellac supplied by Mantrose Haeser Co., Attleboro, Ma.
¹¹Supplied under the tradename Cabosil ® by Cabot, Tuscola, Ill.
¹²Supplied as Mono- and Diglycerides of fats and oils (disposable grade) by Lonza Inc., Fair Lawn, NJ.

In a suitable beaker (beaker A), water, sucralose, potassium phosphate monobasic are added with mixing until the mixture is homogenous.

In a separate beaker (beaker B), xanthan gum, locust bean gum, carrageenan, pullulan and Plasdone K-90 are mixed as a dry mix until the mixture is homogenous. The contents of beaker B are mixed into beaker A with rapid mixing or stirring. The combined mixture is mixed until the gums are hydrated. To the combined mixture, the hydrogen peroxide is added slowly with mixing.

In a separate beaker (beaker C), the flavor, polysorbate 80, glycerin and Atmos 300 are mixed until dissolved and uniform. The contents of beaker C are then poured into beaker A and mixed until the mixture is uniform and homogenous. The pH is then adjusted to about 5.5 using 1.0 N sodium hydroxide.

In still another separate beaker (beaker D), the pharmaceutical glaze, Cabosil, alcohol and glyceryl sterate is mixed until uniform and homogenous.

The contents of beaker D is then cast at desired thickness on a non-stick at room temperature to form the inventive film or first layer of the bi-layer, teeth whitening film.

The contents of beaker A is then cast at desired thickness over the above-described first layer at room temperature to form the second layer of the bi-layer, teeth whitening film.

### Example V

The following is an example of a bi-layer, teeth whitening film of the present invention.

| | |
|---|---|
| | AMOUNT |
| INGREDIENT | (weight percent) |

Adhesive Layer

| | | |
|---|---|---|
| XANTHAN GUM¹ | 0.02308% | w/w |
| LOCUST BEAN GUM, CLARIFIED² | 0.04616% | w/w |
| CARRAGEENAN³ | 0.2308% | w/w |
| POVIDONE, USP K-90⁴ | 16.426% | w/w |
| SUCRALOSE⁵ | 0.7000% | w/w |
| POTASSIUM PHOSPHATE MONOBASIC NF | 0.0700% | w/w |
| PURIFIED WATER, USP/EP | 72.4948% | w/w |
| HYDROGEN PEROXIDE 35%⁶ | 5.7100% | w/w |
| FLAVOR | 2.5890% | w/w |
| POLYSORBATE 80 NF/EP⁷ | 0.3550% | w/w |
| EMULSIFIER⁸ | 0.3550% | w/w |
| GLYCERIN USP SPECIAL | 1.0000% | w/w |

Backing Layer

| | | |
|---|---|---|
| PHARMACEUTICAL GLAZE, 4-LB CUT NF⁹ | 55.0000% | w/w |
| SILICA¹⁰ (fumed untreated) | 4.0000% | w/w |
| ALCOHOL USP/EP | 40.0000% | w/w |
| GLYCERYL STEARATE SE¹¹ | 1.0000% | w/w |

¹Supplied under the name Keltrol T by CP Kelco, Chicago, IL
²Sold under the name Viscogum BCR 20/80 by Degussa Texturant Systems, Atlanta, GA
³Supplied under the name Viscarin SD339 by FMC Biopolymer, Philadelphia, PA.
⁴Polyvinylpyrrolidone, USP K-90, International Specialties Products(ISP), Wayne, NJ.
⁵ALB CG 35% hydrogen peroxide solution, Atofina, Philadelphia, Pa.
⁶Supplied under the tradename Spenda ®, by McNeil Pharmaceuticals, Philadelphia, Pa.
⁷Tween 80, supplied by Quest, _Hoffmann Estates, Ill.
⁸Mixture of mono- and di-oleates supplied under name Atmos 300 by American Ingredients, Kansas City, Mo.
⁹Shellac supplied by Mantrose Haeser Co., Attleboro, Ma.
¹⁰Supplied under the tradename Cabosil ® by Cabot, Tuscola, Ill.
¹¹Supplied as Mono- and Diglycerides of fats and oils (disposable grade) by Lonza Inc., Fair Lawn, NJ.

In a suitable beaker (beaker A), water, sucralose, potassium phosphate monobasic are added with mixing until the mixture is homogenous.

In a separate beaker (beaker B), xanthan gum, locust bean gum, carrageenan and Plasdone K-90 are mixed as a dry mix until the mixture is homogenous. The contents of beaker B are mixed into beaker A with rapid mixing or stirring. The combined mixture is mixed until the gums are hydrated. To the combined mixture, the hydrogen peroxide is added slowly with mixing.

In a separate beaker (beaker C), the flavor, polysorbate 80, glycerin and Atmos 300 are mixed until dissolved and uniform. The contents of beaker C are then poured into beaker A and mixed until the mixture is uniform and homogenous. The pH is then adjusted to about 5.5 using 1.0 N sodium hydroxide.

In still another separate beaker (beaker D), the pharmaceutical glaze, Cabosil, alcohol and glyceryl sterate is mixed until uniform and homogenous.

The contents of beaker D is then cast at desired thickness on a non-stick at room temperature to form the inventive film or first layer of the bi-layer, teeth whitening film.

The contents of beaker A is then cast at desired thickness over the above-described first layer at room temperature to form the second layer of the bi-layer, teeth whitening film.

### Example VI

The following is an example of a bi-layer, teeth whitening film of the present invention.

| | |
|---|---|
| | AMOUNT |
| INGREDIENT | (weight percent) |

Adhesive Layer

| | | |
|---|---|---|
| XANTHAN GUM¹ | 0.0674% | w/w |
| LOCUST BEAN GUM, CLARIFIED² | 0.0848% | w/w |
| PULLULAN³ | 4.1740% | w/w |
| POVIDONE, USP K-90⁴ | 12.4000% | w/w |
| SUCRALOSE⁵ | 0.7000% | w/w |
| POTASSIUM PHOSPHATE MONOBASIC NF | 0.0700% | w/w |
| PURIFIED WATER, USP/EP | 72.4948% | w/w |
| HYDROGEN PEROXIDE 35%⁶ | 5.7100% | w/w |
| FLAVOR | 2.5890% | w/w |
| POLYSORBATE 80 NF/EP⁷ | 0.3550% | w/w |
| EMULSIFIER⁸ | 0.3550% | w/w |
| GLYCERIN USP SPECIAL | 1.0000% | w/w |

Backing Layer

| | | |
|---|---|---|
| PHARMACEUTICAL GLAZE, 4-LB CUT NF⁹ | 55.0000% | w/w |
| SILICA¹⁰ (fumed untreated) | 4.0000% | w/w |
| ALCOHOL USP/EP | 40.0000% | w/w |
| GLYCERYL STEARATE SE¹¹ | 1.0000% | w/w |

¹Supplied under the name Keltrol T by CP Kelco, Chicago, IL
²Sold under the name Viscogum BCR 20/80 by Degussa Texturant Systems, Atlanta, GA
³PI-20 grade supplied by Hayashibara.
⁴Polyvinylpyrrolidone, USP K-90, _ International Specialties Products(ISP), Wayne, NJ.
⁵ALB CG 35% hydrogen peroxide solution, Atofina, Philadelphia, Pa.
⁶Supplied under the tradename Spenda ®, by McNeil Pharmaceuticals, Philadelphia, Pa.
⁷Tween 80, supplied by Quest, _Hoffmann Estates, Ill.
⁸mixture of mono- and di-oleates supplied under name Atmos 300 by American Ingredients, Kansas City, Mo.
⁹Shellac supplied by Mantrose Haeser Co., Attleboro, Ma.
¹⁰Supplied under the tradename Cabosil ® by Cabot, Tuscola, Ill.
¹¹Supplied as Mono- and Diglycerides of fats and oils (disposable grade) by Lonza Inc., Fair Lawn, NJ.

In a suitable beaker (beaker A), water, sucralose, potassium phosphate monobasic are added with mixing until the mixture is homogenous.

In a separate beaker (beaker B), xanthan gum, locust bean gum, pullulan and Plasdone K-90 are mixed as a dry mix until the mixture is homogenous. The contents of beaker B are mixed into beaker A with rapid mixing or stirring. The combined mixture is mixed until the gums are hydrated. To the combined mixture, the hydrogen peroxide is added slowly with mixing.

In a separate beaker (beaker C), the flavor, polysorbate 80, glycerin and Atmos 300 are mixed until dissolved and uniform. The contents of beaker C are then poured into beaker A and mixed until the mixture is uniform and homogenous. The pH is then adjusted to about 5.5 using 1.0 N sodium hydroxide.

In still another separate beaker (beaker D), the pharmaceutical glaze, Cabosil, alcohol and glyceryl sterate is mixed until uniform and homogenous.

The contents of beaker D is then cast at desired thickness on a non-stick at room temperature to form the inventive film or first layer of the bi-layer, teeth whitening film.

The contents of beaker A is then cast at desired thickness over the above-described first layer at room temperature to form the second layer of the bi-layer, teeth whitening film.

### Example VII

The following is an example of a bi-layer, teeth whitening film of the present invention.

| | |
|---|---|
| | AMOUNT |
| INGREDIENT | (weight percent) |

Adhesive Layer

| | | |
|---|---|---|
| STARCH GUM¹ | 1.9674% | w/w |
| GUM ARABIC² | 0.1848% | w/w |
| PULLULAN³ | 2.1740% | w/w |
| POVIDONE, USP K-90⁴ | 12.4000% | w/w |
| SUCRALOSE⁵ | 0.7000% | w/w |
| POTASSIUM PHOSPHATE MONOBASIC NF | 0.0700% | w/w |
| PURIFIED WATER, USP/EP | 72.4948% | w/w |
| HYDROGEN PEROXIDE 35%⁶ | 5.7100% | w/w |
| FLAVOR | 2.5890% | w/w |
| POLYSORBATE 80 NF/EP⁷ | 0.3550% | w/w |
| EMULSIFIER⁸ | 0.3550% | w/w |
| GLYCERIN USP SPECIAL | 1.0000% | w/w |

Backing Layer

| | | |
|---|---|---|
| PHARMACEUTICAL GLAZE, 4-LB CUT NF⁹ | 55.0000% | w/w |
| SILICA¹⁰ (fumed untreated) | 4.0000% | w/w |
| ALCOHOL USP/EP | 40.0000% | w/w |
| GLYCERYL STEARATE SE¹¹ | 1.0000% | w/w |

¹Supplied under the trade name of Pure-Cote B760, supplied by Grain processing Corporation, Muscatine, IA.
²Supplied under the name Bright Gum Arabic Spray Dry FCC/NF Powder by TIC Gums, Belcamp, MD
³PI-20 grade supplied by Hayashibara.
⁴Polyvinylpyrrolidone, USP K-90, International Specialties Products(ISP), Wayne, NJ.
⁵ALB CG 35% hydrogen peroxide solution, Atofina, Philadelphia, Pa.
⁶Supplied under the tradename Spenda ®, by McNeil Pharmaceuticals, Philadelphia, Pa.
⁷Tween 80, supplied by Quest, Hoffmann Estates, Ill.
⁸mixture of mono- and di-oleates supplied under name Atmos 300 by American Ingredients, Kansas City, Mo.
⁹Shellac supplied by Mantrose Haeser Co., Attleboro, Ma.
¹⁰Supplied under the tradename Cabosil ® by Cabot, Tuscola, Ill.
¹¹Supplied as Mono- and Diglycerides of fats and oils (disposable grade) by Lonza Inc., Fair Lawn, NJ.

In a suitable beaker (beaker A), water, sucralose, potassium phosphate monobasic are added with mixing until the mixture is homogenous.

In a separate beaker (beaker B), starch gum, gum arabic, pullulan and Plasdone K-90 are mixed as a dry mix until the mixture is homogenous. The contents of beaker B are mixed into beaker A with rapid mixing or stirring. The combined mixture is mixed until the gums are hydrated. To the combined mixture, the hydrogen peroxide is added slowly with mixing.

In a separate beaker (beaker C), the flavor, polysorbate 80, glycerin and Atmos 300 are mixed until dissolved and uniform. The contents of beaker C are then poured into beaker A and mixed until the mixture is uniform and homogenous. The pH is then adjusted to about 5.5 using 1.0 N sodium hydroxide.

In still another separate beaker (beaker D), the pharmaceutical glaze, Cabosil, alcohol and glyceryl sterate is mixed until uniform and homogenous.

The contents of beaker D is then cast at desired thickness on a non-stick at room temperature to form the inventive film or first layer of the bi-layer, teeth whitening film.

The contents of beaker A is then cast at desired thickness over the above-described first layer at room temperature to form the second layer of the bi-layer, teeth whitening film.

1. A film composition comprising:
a.) at least one water insoluble polymer;
b.) at least one disintegration facilitator selected from the group consisting of water insoluble particulates, plasticizer and mixtures thereof.
c.) optionally, at least one topical or systemic active
wherein the film is disintegratable in an aqueous environment.

2. A film composition according to claim 1 wherein the water insoluble polymer is selected from the group consisting of hydrogenated vegetable oils, hydrogenated caster oil, polyvinyl chloride, shellac, polyurethane, cellulose derivatives, gum rosens, wood rosens, waxes, acrylate and methacrylate polymers, copolymers of acrylic and methacrylic acid esters or mixtures thereof.

3. A film composition according to claim 2 wherein the water insoluble polymer is shellac.

4. A film composition according to claim 1 wherein the plasticizer is selected from the group consisting of citric acid alkyl esters, glycerol esters, phthalic acid alkyl esters, sebacic acid alkyl esters, sucrose esters, sorbitan esters, acetylated monoglycerides, glycerols, glycols, fatty acid esters, propylene glycol, poloxamers, alkyl aryl phosphates and polyethylene glycols 200 to 12,000 and mixtures thereof.

5. A film composition according to claim 4 wherein the plasticizer is glycerol monostearate.

6. A film composition according to claim 1 wherein the water insoluble particulate is selected from the group consisting of alumina, talc, titanium dioxide, magnesium stearate, barium titanate, magnesium titanate, calcium titanate, strontium titanate, zinc oxide, silica sand, clay, mica, tabular spar, diatomaceous earth, various inorganic oxide pigments, chromium oxide, cerium oxide, iron red, antimony trioxide, magnesium oxide, zirconium oxide, barium sulfate, barium carbonate, calcium carbonate, silica, silicon carbide, silicon nitride, boron carbide, tungsten carbide, titanium carbide, carbon black and mixtures thereof.

7. A film composition according to claim 6, wherein the water insoluble polymer is fumed silica.

8. A film composition according to claim 1, further comprising at least one topical or systemic active.

9. A film composition according to claim 1 wherein the topical or systemic active is selected from the group consisting of whitening agents, antitartar agents, fluoride ion sources, antimicrobial agents, antiinflammatory agents, upper respiratory agents, gastrointestinal agents, enzymes, antifungals, antibiotics, analgesics, histamine antagonists and mixtures thereof.

10. A multi-layer film composition comprising at least two film layers wherein at least one layer comprises:
a.) at least one water insoluble polymer;
b.) at least one disintegration facilitator selected from the group consisting of water insoluble particulates, plasticizers and mixtures thereof; and
c.) optionally, at least one topical or systemic active
wherein the film is disintegratable in an aqueous environment.

11. A film composition according to claim 10, further comprising at least one topical or systemic active.

12. A film composition according to claim 11 wherein the topical or systemic active is selected from the group consisting of whitening agents, antitartar agents, fluoride ion sources, antimicrobial agents, antiinflammatory agents, upper respiratory agents, gastrointestinal agents, enzymes, antifungals, antibiotics, analgesics, histamine antagonists and mixtures thereof.

13. A film composition according to claim 12, wherein the topical or systemic active is a whitening agent.

14. A film composition according to claim 13, wherein the whitening agent is selected from the group consisting of peroxides, metal chlorites, perforates, percarbonates, peroxyacids, and mixtures thereof.

15. A film composition according to claim 14, wherein the whitening agent is hydrogen peroxide.

16. A method for treating the skin, oral mucosa or teeth comprising the step of applying to such area(s) the film composition of claim 1.
